(19) United States
(12) Patent Application Publication
(10) Pub. No.: US 2003/0212129 A1
(43) Pub. Date: Nov. 13, 2003

(54) System and method for revitalizing human skin

(76) Inventors: Kay Miyakawa Liu, Somerset, CA (US); Craig Miller, Diamond Bar, CA (US)

Correspondence Address: Craig Miller, 23719 Jayhawker, Diamond Bar, CA, 91765, US

(21) Appl. No.: 10/143,915

(22) Filed: May 13, 2002

(51) Int. Cl. A61K 31/375; A61N 1/30

(52) U.S. Cl. 514/474; 604/20

(57) ABSTRACT

The present invention is directed to a system and method that revitalizes the skin and effectively restoring the skinks natural pigmentation. The system utilizes the Sonophoresis process to effectively supply vitamin C to Keratinocyte cells located in the epidermis layers of the skin. The readily available vitamin C enables the skin to restore the Keratinocyte cells to natural state by stabilizing the levels of melanin that are stored within the Keratinocyte cells. Once normal levels of melanin, which are stored in the Keratinocyte cells, are achieved the pigmentation of the skin is lightened and restored to a natural color.

### FIELD OF THE INVENTION

The present invention relates to revitalizing layers of the skin or other biological membrane material. In the method a chemical system is delivered to the layers of the skin by use of an ultrasonic device. Upon delivery of the chemical system past the dermal layers the system works with the metabolism to regenerate the skin.

### BACKGROUND OF THE INVENTION

Methods of passing a substance through the skin and into the blood stream are well-known in the art of practicing medicine. In general, Sonophoresis, or injecting a substance through the skin is a well-known method of introducing a substance to the blood stream. In general, the use of chemical enhancers, i.e., wherein a substance is introduced to the skin with a penetration enhancer, which is combined with the substance to ease the penetration of the skin.

Furthermore, users of cosmetic and dermatological products are increasingly seeking to lighten and rejuvenate the skin. The attempts of lightening the skin are usually caused by the desire to stabilize the overall pigmentation of the skin where a user may have one area of the skin, which is darker in color. Skin rejuvenation and lightening are not only due to age but also to exposure to the sun or other radiation. There have been many attempts to rejuvenate and lighten the skin. Most common practices use a lotion form of various vitamins and minerals i.e. vitamin C which is well-known in the art to stimulate the human body's metabolism in a localized area thereby causing the human body to repair and heal. Unfortunately, these commonly known lotions are applied topically, above the Epidermis, and do not affectively apply vitamins and minerals to the most critical layer of the skin the Dermis layer which is below the Epidermis.

### SUMMARY OF THE INVENTION

The present invention provides a method for lightening and rejuvenating the skin comprising the steps of using a chemical system which is designed specifically for supplying vitamins and minerals directly to the Dermis layer of the skin and an ultrasonic device which is similar to the devices used in the Sonophoresis process.

Additionally, an advantage of the present invention is to provide a method of utilizing a chemical system and an ultrasonic device to actively deliver the vitamins and minerals provided in the chemical system to the Dermis layer of the skin thereby stimulating the enabling rejuvenation.

### BRIEF DESCRIPTION OF THE DRAWINGS

FIG. 1 illistrates a normal keratinocyte.

FIG. 2 illustrates keratinocyte cells obtaining melanin from the melanocyte cells.

FIG. 3 illistrates the present invention.

### DETAILED DESCRIPTION OF THE INVENTION

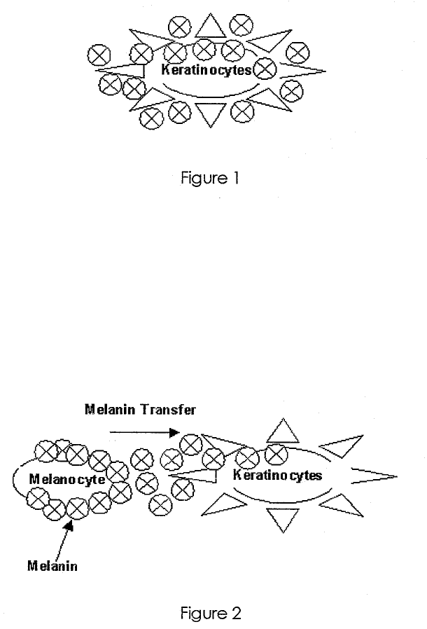

Keratinocytes cells 50 as shown FIG. 1 are a part of the epidermis 60, which is the first barrier at the border of the body. More specifically, Keratinocytes cells 50 aid in the regulation of the bodies skin color to protect the body from harmful ultra violet light. Keratinocytes cells 50 provides this protection by sending signals to the body, which notify the body of harmful conditions.

As shown in FIG. 2, upon notification of harmful conditions the bodies responds by increasing the metabolism thus causing Melanin 70 to excrete from the Melanocyte 80. Melanin 70 is a human's only protection from the natural rays of the sun. Melanin 70 has a high optical density, Which enables it to absorb and scatter impinging ultraviolet radiation. The Melanin 70 is then gathered by the Keratinocytes cells 50 and stored within. Due to Melanin's high optical density, as the storage level of Melanin 70 increases in the Keratinocytes cells 50 the pigmentation of the skin becomes darker.

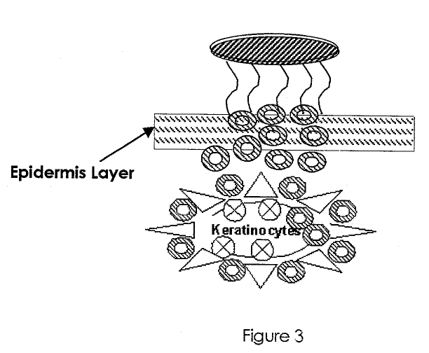

In the representative embodiment of the revitalizing human skin system shown in FIG. 3, an ultrasonic device 30 deposed near or on the surface of the skin and when in operation resonates an ultrasonic wave in a frequency at a magnitude between 1 megahertz to 3 megahertz. As the ultrasonic waves come into contact with the dermis layers of the skin the lipid bilyer begins to absorb the ultrasonic waves. The absorption of the ultrasonic waves causes the cells in the lipid bilayer to move and destabilize thus creating air pockets and allowing small particle sized materials to pass through the layers of the skin. This process is well known in the art as Sonophoresis.

Once the Sonophoresis process enables the pass of small particles ranging from 5 μm to 200 μm by use of an ultrasonic device a liquid form of vitamin C 10 like L-ascorbic acid (L-3-Ketothreohexuronic acid lactone) can be inserted into the epidermis layers 60 of the skin causing the a liquid form of vitamin C to contact with the Keratinocytes cells 50 cells housed in the epidermis layers of the skin. The process of inserting the liquid form of vitamin C to the skin thereby providing vitamin C to the Keratinocyte cells 50 cells is performed by applying the liquid form of vitamin C to the skin thereby forming a coating. Than, manually osculating the ultrasonic device near the skin that contains a coating of a liquid form of vitamin C for duration of time between 1 to 5 minutes. At the point in time where the lipid bylayer (not shown) becomes disorganized the liquid form of vitamin C may pass through the lipid bylayer and into the epidermis layers of the skin thus providing a liquid form of vitamin C to the Keratinocytes cells 50. Vitamin C is an ascorbic acid and operates at a cellular level and provides the function of exporting procollagen molecules like Melanin 70 out of cells. The resulting effect of exporting the Melanin 70 in the Keratinocytes cells 50 is the restoration of the pigmentation of the skin.

Another advantage of providing vitamin C directly to the cells of the skin results in the positive affect vitamin C has on cells and their structures. Vitamin C protects the DNA of the cells from the damage caused by free radicals and mutagens. It prevents harmful genetic alterations within cells and protects lymphocytes from mutations to the chromosomes. Vitamin C may be especially important in this day and age of widespread environmental pollution because it combats the effects of many such toxins, including ozone, carbon monoxide, hydrocarbons, pesticides and heavy metals thus resulting in the restoration of the skin.

What is claimed is:

1. A revitalizing human skin system comprising:
an ultrasonic device capable of producing ultrasonic waves with a frequency at a magnitude between 1 megahertz to 3 megahertz and capable of being deposed near the surface layer of the skin; and
a liquid form of vitamin C which is composed of a particles sizes between 5 μm to 200 μm;
wherein ultrasonic device is capable of destabilizing the lipid bylayer of the skin.

2. The revitalizing human skin system of claim 1, wherein the ultrasonic device is a portable device.

3. The revitalizing human skin system of claim 1, wherein the liquid form of vitimin C is L-ascorbic acid (L-3-Ketothreohexuronic acid lactone).

4. A revitalizing human skin process comprising the steps of
destabilizing the lipid bylayers of the skin using an ultrasonic device, and
inserting liquid forms of vitamin C into the epidermis layers of the skin, and
providing vitamin C to the Keratinocyte cells of located in the epidermis layers of the skin.

5. The revitalizing human skin system of claim 4, wherein the liquid form of vitimin C is L-ascorbic acid (L-3-Ketothreohexuronic acid lactone).

6. A method of revitalizing human skin comprising the steps of
applying a liquid form of vitamin C to the skin thereby forming a coating; and
manually osculating an ultrasonic device near the skin that contains a coating of the liquid form of vitamin C.

7. The revitalizing human skin process of claim 6 wherein the liquid forms of vitamin C is L-ascorbic acid (L-3-Ketothreohexuronic acid lactone).

8. A method of revitalizing human of claim 9 wherein manually osculating an ultrasonic device near the skin for a duration of 1 to 5 minutes.
